(19) United States
(12) Patent Application Publication
(10) Pub. No.: US 2002/0123071 A1
(43) Pub. Date: Sep. 5, 2002

(54) Method of identifying compounds capable of acting as agonists or antagonists of G-protein coupled receptors

(76) Inventors: Sanne Moller Knudsen, Vaerlose (DK); Fritz Poulsen, Hillerod (DK); Anders Heding, Copenhagen (DK)

Correspondence Address: Reza Green, Esq., Novo Nordisk of North America, Inc., Suite 6400, 405 Lexington Avenue, New York, NY, 10174-6401, US

(21) Appl. No.: 10/003,846

(22) Filed: Dec. 4, 2001

Related U.S. Application Data
(60) Provisional application No. 60/251,311, filed on Dec. 5, 2000.

(30) Foreign Application Priority Data
Dec. 4, 2000 (DK) PA 2000 01816

(51) Int. Cl. G01N 33/53

(52) U.S. Cl. 435/7.1

(57) ABSTRACT

The present invention relates to a method for identifying compounds capable of affecting the activity of G-protein coupled receptors (GPCRs), i.e. identifying agonists or antagonists of said receptor type. Such compounds are later useful e.g. as drugs or as leads in drug development. More specifically, if agonists are desired, the method comprises contacting a test compound with cell membrane from a GPCR expressing cell line, a kinase, such as G-protein coupled receptor kinase (GRK), and arrestin. If the test compound is an agonist it will allow said arrestin to bind to activated and phosphorylated GPCR. The above mentioned mixture is then contacted with carrier material capable of binding GPCRs, and signals are detected as an indication that the test compound is capable of acting as an agonist of GPCR. If on the other hand receptor antagonists are desired, the present method is modified to be a competition assay, wherein known agonists are also added to the incubation mixture and compounds are tested as to their capability to displace agonists bound to the receptor, and thereby dissociate arrestin from the receptor. In both of the above described methods, the kinase and the arrestin may be replaced by a phosphorylation independent mutant of the arrestin, omitting the use of a kinase.

### CROSS REFERENCE TO RELATED APPLICATIONS

This application claims priority under 35 U.S.C. 119 of Danish application no. PA and U.S. provisional application 60/251,311 filed on Dec. 5, 2000, the contents of which is fully incorporated herein by reference.

### TECHNICAL FIELD

The present invention relates to a method of identifying compounds which are capable of acting as agonists or antagonists of G-protein coupled receptors (GPCRs). Such compounds may be used as, or further developed into, drugs for activation or deactivation of GPCRs. The present method is advantageously used as a high throughput screening (HTS) method.

### BACKGROUND

In order to identify novel drugs, and specifically leads for novel drugs, assays wherein large amounts of compounds are tested simultaneously for their biological properties are used extensively these days. The most commonly used format is the binding assays, wherein samples are tested for their respective capabilities to displace a labelled known ligand from a receptor under investigation. However, this assay has several major drawbacks. The most important drawback of such competition assays is fact that these assays are not functional assays, and therefore do not allow for any differentiation between agonists and antagonists. This is of great importance when it comes to the study of receptors, the signalling of which is controlled in nature by agonists as well as by antagonists. The second major drawback of the competition assays is that since a receptor specific ligand must be identified, these assays cannot be used for the investigation of orphan receptors, as no known ligand is known for these receptors.

Further, the labelling of the known ligand renders competition assays relatively cumbersome and therefore time consuming. Yet another disadvantage with conventional binding assays is that only compounds with affinity for the same binding site as the known ligand will be discovered and accordingly unknown binding sites on the receptor will not be discovered.

Thus, in order to gain a higher degree of functional knowledge receptors expressed in whole living cells are often studied. Cells are then studied using a focusing microscope, which method however requires much time and precision. A previously labelled sample is added to the microscope slide and an exact adjustment thereof is then required in order to obtain an informative picture. Since the conformation of a living cell is constantly changing with time, a certain skill is required with these methods. Reliable information regarding a receptor's structure, components and in certain cases its interaction with one specific ligand can thereby be obtained. However, whole cell studies using microscope are very time consuming and not convenient when the purpose is to study a large amount of candidate compounds' interactions with a receptor.

U.S. Pat. No. 5,891,646 in the name of Barak et al teaches a method for assaying receptor activity. More specifically, the interaction of a G-protein coupled receptor (GPCR) with β-arrestin conjugated to a detectable molecule is studied within cells and the β-arrestin redistribution from the cytosol to the plasma membrane is disclosed. The purpose of the methods described therein is primarily an understanding of the mechanisms of action of various therapeutic agents. Furthermore, U.S. Pat. No. 5,891,646 also proposes a method for screening for GPCR agonists and antagonists. However, such screening is also based on the use of whole living cells. This is a serious limitation in the screening of large libraries, since whole cells require a great degree of care when deposited on supporting substrates. Accordingly, these methods cannot be efficiently automated and are therefore not applicable in the context of the fast and simple high throughput screening methods, which are the most advantageous when compound libraries are screened.

Accordingly, there is a need within this field of novel methods that enable rapid and convenient screening of large numbers of candidate compounds for their biological effect on G-protein coupled receptors (GPCRs).

### SUMMARY OF THE INVENTION

The object of the present invention is to provide a method for identifying biologically active compounds with a reduced degree of disturbance compared to the conventional binding assays discussed above. Another object of the invention is to provide a method for identifying a biologically active compound, which method enables differentiating between agonists and antagonists. A further object of the invention is to provide a method which is suitable for use in high throughput screening. Accordingly, the method according to the present invention provides the identification of novel drugs and/or drug leads in a faster and more convenient way than the above-discussed assays.

More specifically, the present method provides the identification of a compound, which is capable of initiating the signalling of a G-protein coupled receptor (GPCR). This is achieved by incubating a test compound with a preparation of cell membrane from a GPCR expressing cell line, a kinase and a arrestin, preferably a β-arrestin and preferably labelled. This will allow the arrestin to bind to the GPCR, if the test compound is an agonist and causes the GPCR to be activated and phosphorylated. The GPCR bound arrestin is then separated from the unbound arrestin, for instance by contacting the mixture so obtained contacted with carrier material capable of binding said cell membranes, and the levels of GPCR bound arrestin are determined (or the signals emitted from the formed (arrestin-GPCR) complex, or (arrestin-GPCR-carrier) complex detected). The level of GPCR bound arrestin may be detected in a number of ways known to the person skilled in the art. The level of GPCR bound arrestin may for instance be determined by labelling the arrestin and by using a carrier material capable of interacting with said labelling, the nature of which determine how the emitted signals for determining the level of GPCR bound arrestin are detected. For instance may signals emitted from the carrier material or the labelled arrestin be detected by any method, such as detection of light emitted or detection of radioactivity, e.g. by use of a carrier material comprising scintillation proximity assay (SPA) beads (Cook, N. D., Drug Discovery Today 1(7), 287-294 (1996): Scintillation proximity assay—a versatile high throughput screening technology) etc. Such signals are then an indication of presence of an agonist in the sample. Other embodiments and aspects are as defined by the appended claims, and more details will be given below.

### DETAILED DESCRIPTION OF THE INVENTION

In a first aspect the present invention relates to a method of identifying a compound capable of initiating the signalling of a G-protein coupled receptor (GPCR), which method comprises

(a) contacting at least one test compound with a preparation of cell membrane from at least one GPCR expressing cell or cell line, at least one kinase, and at least one arrestin in a suitable buffer;

(b) separating the GPCR bound arrestin from the unbound arrestin; and

(c) determining the level of GPCR bound arrestin,

whereby a compound, which is an agonist of said GPCR, is identified when the level of GPCR bound arrestin is raised relative to a control.

The contact between at least one test compound, cell membrane from at least one GPCR expressing cell or cell line, at least one kinase and at least one arrestin takes place in a suitable buffer. Such a buffer can be any buffer, which provides conditions suitable for activation of GPCR present in the preparation of cell membrane, such as for instance a buffer comprising 50 mM Tris-HCl, pH 7.5, 50 mM potassium acetate, 0.5 mM MgCI2, 1 mM DTT, and 0.5 mM ATP. How to determine GPCR activation and suitable assay conditions for activation of GPCRs are well known in the field. Activation of GPCRs may for instance be determined by measuring the level of cAMP, Ca²⁺ or via the ³⁵S-GTPγS assay for determining GTP binding of G protein (for instance in the assay Adenylyl Cyclase Activation made by FlashPlate Assay by Perkin Elmer—see also “Signal Transduction”—A practical approach”, editor G. Milligan, IRL Press Oxford (1992). If a significant difference is determined in these assays, the GPCR is considered to be activated. The activation of a GPCR is the event that initiates the signal transduction pathway.

In the present context, the term “contacting” is intended to encompass any incubating necessary in order to get the kinase to phosphorylate the GPCR and to allow the arrestin to bind to activated and phosphorylated GPCR. The time span for such an incubation may be typically between about 15 and 120 minutes, such as about 30 minutes or 60 minutes, but the necessary incubation time can be shorter or longer. It may even be that the arrestin binds the activated GPCR during step (a) so fast that the allowance of incubation time for the binding to take place will not be necessary. However, it is necessary that the arrestin is allowed to bind to activated GPCR in any method according to the present invention whether or not a specific incubation time period is included in said method. In addition, there should also be allowed time for the test compound to activate the GPCR. However, as it is clear from the above, the allowance of a specific incubation period may not always be necessary.

GPCR bound arrestin shall mean arrestin which have bound the GPCR in the cell membrane with a K_{D} which is small enough to allow the separation of GPCR bound arrestin from unbound GPCR arrestin. The K_{D} will normally be lower than 10⁻⁶ M, such as for instance lower than 10⁻⁷ M, such as for instance lower than or even as low as about 10⁻¹² M. As an example, β-arrestin binds the B2-adrenergic receptor with a K_{D} of 1,8 nM. As it will become clear from some of the further embodiments of the method according to the present invention a K_{D} of the binding of arrestin to the GPCR small enough for a label on the bound arrestin to interact with the specific carrier material, which might be attached to the cell membranes, is also encompassed in the present invention. An example of a direct binding assay can be found in Kovoor et al., Journal of Biological Chemistry 274, 6831-6834 (1999).

The separation of the GPCR bound arrestin from the unbound arrestin can be performed by well-known methods in the field, such as centrifugation, precipitation and filtration. The GPCR—or the membranes in which they are embedded—may also be attached to a solid carrier and then the unbound arrestin can be simply “washed off” the GPCR bound arrestin.

Methods for determining the level, or the amount, of GPCR bound arrestin are well known in the art. The amount of GPCR bound arrestin can for instance be determined by use of SDS-page, Western Blots or other methods for determining amount of protein and then compared to the level of GPCR bound arrestin in a control, wherein vehicle or a known agonist are used instead of a test compound. In particular, the arrestin may be labelled as described in more detail below, and the amount of label bound to the cell membrane, of which GPCRs are an integral part, can be measured by use of methods, the nature of which depend on the nature of the label.

An agonist is identified when the level of GPCR (or membrane) bound arrestin is significantly higher in a method according to the invention using a test compound compared to the level of GPCR (or membrane) bound arrestin in such a method using vehicle, that is, no test compound under similar conditions. The significance of a higher level may be determined by statistical methods and/or by the specific nature and accuracy of the determining technique as it is well known in the art.

Thus, as appears from the above, contrary to conventional binding assays, the present method can advantageously be used with orphan receptors, and it is also easily adapted to an automated procedure due to the technical simplicity thereof.

G-protein coupled receptors (GPCRs) represent a large superfamily of proteins that transduce extracellular signals to the interior of cells, wherein each individual GPCR type activates a particular signal transduction pathway. Several different signal transduction pathways are hitherto known to be activated via GPCRs. For example adrenergic receptors, such as the β2-adrenergic receptors, which is a prototype mammalian GPCR, Dopamine D1a receptor, NMDA receptor, the GLP-1 receptor, the GLP-2 receptor, somatostatin receptors, serotonin receptors, opioid receptors, neuropeptide Y receptors, H3 receptors, galanin receptors, angiotensin receptors, follicle stimulating hormone receptor, prostaglandin receptors, GABA B receptors and orphan GPCRs or any other GPCR may be used in a method according to the present invention.

Signalling through GPCRs rapidly desensitizes, primarily as the consequence of receptor phosphorylation, even though receptor sequestration and down-regulation may also contribute to this process. Two families of serine/threonine kinases, second messenger dependent protein kinases and receptor-specific G-protein coupled receptor kinases, phosphorylate GPCRs and thereby contribute to receptor desensitising. Receptor-specific phosphorylation of GPCRs promotes the binding of cytosolic proteins referred to as arrestins, which function to further uncouple GPCRs from the heterotrimeric G-proteins. An overview of GPCRs and their signalling mechanisms can be found in H. Bourne, Current Biology 9, 134-142 (1997) and E. J. M. Helmreich, Biochimica et Biophysica Acta 1286, 285-322 (1996). Both the kinase and the arrestin used in the present method will be discussed in more detail below.

The present test compounds are preferably obtained from libraries and may be peptides or other organic molecules. The test compounds may be organized according to standard methods into systems or arrays enabling a systematic testing of possible variations of chemical compositions such that several test compounds are contacted with cell membrane preparation in the same vessel. Chemical libraries, such as combinatorial chemical libraries, comprise chemical compounds that have been synthesized from a systematic series of reactions. Such libraries can include an extraordinarily large and varied collection of compounds. It is obvious for a person skilled in the art that the methods according to the invention may also be used to make controls, that is without the addition of a test compound, to give a basis for for instance determining the basal levels of GPCR bound arrestin and for other purposes as well as using a known GPCR agonist or antagonist instead of a test compound for for instance comparison purposes and also any other form for controls that might be relevant as control for said test compound.

A “preparation of cell membrane from at least one GPCR expressing cell or cell line” or “cell membrane from at least one GPCR expressing cell line” is a composition comprising GPCR or GPCRs as integral parts of a cell membrane, but in a form that makes both faces of the membrane accessible to any added compounds. Cell membranes from GPCR expressing cells or cell lines are prepared from cells capable of expressing the GPCR under investigation. More specifically, the present cell membrane may be from eukaryotic or prokaryotic cells, such as bacterial cells, for instance Eschericia coli, yeast cells, for instance Saccharomyces cerevisiae, fungal cells, for instance Aspergillius niger, insect cells, for instance Sf9 cells, nematode cells, plant or animal cells. Examples of animal cells are mammalian cells, such as HEK cells, HeLa cells, COS cells, CHO cells, BHK cells and various primary mammalian cells. The cells may express the GPCR endogenously or may express the GPCR under investigation as a result of genetic engineering.

Methods for preparing cell membrane from a cell or a cell line are well known in the field. Methods for preparing cell membrane from bacterial cells, which often express integral membrane proteins in inclusion bodies, may comprise using an anionic agent to enable the extraction of expressed GPCRs from inclusion bodies in a form, which can be reconstituted into membrane preparations. Depending on the purpose of the investigation the cell, such as a cultured cell line or a cell isolated from a human being, is disintegrated into fragments of suitable sizes by methods, such as French press, sonication, physical homogenisation etc. The use of anionic agents may also be used in methods for preparing cell membrane from prokaryotic cells. When preparing cell membrane from mammalian cells, the membranes may be fused. The fusion of biological membranes or lipid bilayers is the process by which the two surfaces meet en face and reform to yield a new surface, on which molecules on either membrane that were oriented toward the contacting faces become oriented in the same direction. After fusion, components of each original membrane have the potential to mix with the lipid and protein components of the other. A protocol for the membrane preparation and the two-part reconstitution (membrane fusion) of plasma membrane are for instance described in Cerione et al., Nature 306, 562-566 (1983). PEG, Ca²⁺ and electrofusion have been the fusogen techniques of greatest utility in this protocol as described by Schramm et al., Proc.Natl.Acad.Sci.USA 76, 1174-1178 (1979) and Zimmermann and Vienken, J. Membrane Biol. 67, 165-182 (1982). Examples of detergents for use in such a protocol or generally in preparing preparation of cell membrane comprise digitonin. saponin, n-octylglucoside, n-dodecylglucoside, n-dodecylmaltoside, octanoyl-N-methylglucamide, decanoyl-N-methyl-glucamide, Triton® X-100, Triton® X-114, Thesit®, isotridecypoly(ethylene glycol ether)ₙ, 3-[(3-cholamidopropyl)dimethylamminio]-1-propane sulphonate (CHAPS), 3-[(3-cholamidopropyl)dimethylamminio]-2-hydroxy-1-propane sulphonate (CHAPSO) and N-dodecyl=N,N-dimethyl-3-amminio-1-propane sulphonate.

In a method according to the present invention, the kinase may be any kinase capable of phosphorylating a given GPCR. It may for instance be a G-protein coupled receptor kinase (GRK), a second messenger dependent protein kinase, or any other kinase capable of phosphorylating the GPCR. To date, the GRK protein family consists of six members, which can be further classified into subgroups according to sequence homology and functional similarities, and any one of these, such as GRK-1 or GRK-2, may be used in the present context (for a review of GRKs, see Palczewski, Eur. J. Biochem. 248, 261-269 (1997)). The person skilled in this field will realise that various combinations of GPCR-kinase may be tested in order to determine an optimal enzyme for the receptor used. In one embodiment of a method according to the present invention, the kinase is a G-protein coupled receptor kinase (GRK), such as GRK-2.

Arrestin and β-arrestin are well known proteins which occur in nature in several forms, all of which are contemplated in the present context. The function of arrestin in nature is to deactivate a GPCR by binding to its activated and phosphorylated form. Such binding causes the receptor signalling to stop and/or the receptor to internalize. In a method according to the present invention, the term arrestin shall encompass all types of wild-type arrestins and mutants and derivatives of wild-type and mutant arrestins as long as these mutants and derivates maintain the ability to bind the GPCR. Examples of derivatives of arrestin may for instance be arrestin molecules fused to other polypeptides or arrestin molecules in which one or more amino acid residues are modified for instance by acylation or glycosylation. Some mutants and derivatives may have the ability to bind GPCRs independently of for instance the activation/phosphorylation state of the GPCR (see for instance Kovoor (1999), above, and such mutants are also encompassed in the present invention as well as other mutants with specific or different requirements for binding. Similarly, the terms “(arrestin-GPCR) complex” and “(arrestin-GPCR-carrier) complex” shall also encompass any complexes comprising β-arrestin or any arrestin mutants. The arrestin for use in a method according to the present invention may thus for instance be a β-arrestin and may e.g. be selected from the group consisting of β-arrestin 1, β-arrestin 2, β-arrestin 3 and β-arrestin 4 (for a general reference to arrestins, see e.g. Gurevich, Journal of Biological Chemistry 270, 720-731 (1995)) or mutants or derivatives thereof. Mutants and derivatives of other arrestins are also encompassed by the present invention. In one embodiment of a method according to the present invention, the arrestin is β-arrestin.

Arrestin (including arrestin mutants) according to the present invention may for instance be prepared by isolation them from their natural environment or by use of well-known recombinant techniques. Arrestins can for instance be prepared by placing a recombinant expression vector carrying a gene encoding said arrestin under conditions, which results in expression of the recombinant arrestin protein. Such conditions may for instance be the conditions inside a cell, but may also be established in vitro (Kovoor (1999), above). Cells which are capable of providing such conditions may be eukaryotic or prokaryotic cells, such as bacterial cells, for instance Eschericia coli, yeast cells, for instance Saccharomyces cerevisiae, Saccharomyces kluyveri, Schizosaccharomyces pombe, Kluyveromyces lactis, Hansenula polymorpha, and Candida sp., fungal cells, for instance Aspergillius niger insect cells, for instance Sf9 cells, nematode cells, plant or animal cells. Examples of animal cells are mammalian cells, such as HEK cells, HeLa cells, COS cells, CHO cells, BHK cells and various primary mammalian cells. Such methods are especially useful for arrestin mutant proteins. The purification of the arrestin may be performed by use of methods well known in the art.

In one embodiment, the method described above is modified by the use of a mutant arrestin, wherein the mutation renders the arrestin phosphorylation independent. Thereby the addition of kinase, such as G-protein coupled receptor kinase (GRK), can be excluded. Thus, such a method will comprise the following steps:

(a) contacting at least one test compound with cell membrane from at least one GPCR expressing cell or cell line and at least one phosphorylation independent arrestin mutant

(b) separating the GPCR bound arrestin mutant from the unbound arrestin mutant; and

(c) determining the level of GPCR bound arrestin,

whereby a compound, which is an agonist of said GPCR, is identified when the level of GPCR bound arrestin mutant is raised relative to a control.

The mutant arrestin may be a mutant β-arrestin, which may e.g. be selected from R169E (where amino acid number 169, arginine, is changed to glutamic acid) or 1-382-β-arrestin (where the β-arrestin has been truncated and only contains the first 382 amino acids) (see e.g. Krupnic, Annu. Rev. Pharmacol. Toxicol. 38, 289-319 (1998); and Kovoor (1999), above).

However, as the person skilled in this field will realize, novel useful phosphorylation independent mutants of different arrestins, such as β-arrestin, may be created, the use of which are also within the scope of the present invention. In addition, the present invention also encompasses the use of any otherwise modified arrestin, such as β-arrestin, e.g. a protein from which one or more amino acids have been removed, or subunits of a arrestin, such as β-arrestin, as long as the modified arrestin is capable of exerting its function of binding to activated GPCR in the present method.

The further details regarding this embodiment are as discussed above. When a phosphorylation independent arrestin is used in absence of a kinase in a method according to the present invention, the term “contacting” is intended to encompass any incubating necessary in order to allow the arrestin mutant to bind to activated GPCR. The time span for such an incubation may be typically between about 15 and 120 minutes, such as about 30 minutes or 60 minutes, but the necessary incubation time can be shorter or longer. It may even be that the arrestin mutant binds the activated GPCR during step (a) so fast that the allowance of incubation time for the binding to take place will not be necessary. However, it is necessary that the arrestin mutant is allowed to bind to activated GPCR in any method according to the present invention whether or not a specific incubation time period is included in said method. In addition, there should also be allowed time for the test compound to activate the GPCR. However, as it is clear from the above, the allowance of a specific incubation period may not always be necessary.

Some phosphorylation independent arrestin mutants will nevertheless bind even stronger to the GPCR when a kinase is present (Kovoor (1999), above). Including a kinase, such as a G-protein coupled receptor kinase (GRK), for example GRK-2, in step (a) in a method according to the present invention modified by the use of a mutant arrestin, wherein the mutation renders the arrestin phosphorylation independent, will thus allow for an assay with an even lower signal/noise ratio, as the positive response will be stronger.

A method according to the present invention modified by the use of at least one phosphorylation independent arrestin mutant, wherein the test compound in step (a) is furthermore brought into contact with a kinase is thus also encompassed in the present invention. In one embodiment of such a method, the kinase is a G-protein coupled receptor kinase (GRK), such as GRK-2.

In one embodiment of a method according to the present invention, the separation of the GPCR bound arrestin from the unbound arrestin is achieved by use of a carrier material capable of binding the cell membranes. Such a method will thus comprise the following steps:

(a) contacting at least one test compound with cell membrane from at least one GPCR expressing cell or cell line; at least one kinase; and at least one arrestin in a suitable buffer;

(b) contacting the resulting mixture with carrier material capable of binding said cell membrane(s); and

(c) determining the level of GPCR bound arrestin,

whereby a compound, which is an agonist of said GPCR, is identified when the level of GPCR bound arrestin is raised relative to a control.

The carrier material provides separation of GPCR bound arrestin from unbound arrestin by binding the (arrestin-GPCR) complex formed during the incubation step and may be any kind of support well known in this field.

In one embodiment, the carrier material is in the form of beads, which are covered by a suitable binding partner to membranes capable of containing a GPCR, such as wheat germ agglutinate (WGA), antibodies against the receptor or antibodies against one or more inserted recognition sites in the receptor. The preparation of an antibody raised against a known protein or peptide sequence is easily performed by the person skilled in this field.

Thus, determining the level of GPCR bound arrestin in a method according to the present invention is uncomplicated, fast and efficient, since steps for separating bound arrestin from unbound arrestin are made easier.

The further details regarding this embodiment are as discussed above. In particular, said kinase may be a G-protein coupled receptor kinase (GRK), such as GRK-2, as described above and/or said arrestin may be β-arrestin.

In one embodiment, a method comprising the use of a carrier material as described above is modified by the use of a mutant arrestin, wherein the mutation renders the arrestin phosphorylation independent as described above. Such a method will thus comprise the following steps:

(a) contacting at least one test compound with cell membrane from at least one GPCR expressing cell or cell line and at least one phosphorylation independent arrestin mutant in a suitable buffer;

(b) contacting the resulting mixture with carrier material capable of binding said cell membrane(s); and

(c) determining the level of GPCR bound arrestin,

whereby a compound, which is an agonist of said GPCR, is identified when the level of GPCR bound arrestin mutant is raised relative to a control.

The further details regarding this embodiment, are as discussed above. The details regarding the use of a mutant arrestin, wherein the mutation renders the arrestin phosphorylation independent, are also as described above. In particular, the arrestin mutant may be a β-arrestin mutant, which in turn may be R169E-β-arrestin or 1-382-0-arrestin as described above, and the test compound in step (a) may also furthermore be brought into contact with a kinase for example a G-protein coupled receptor kinase (GRK), such as GRK-2.

In a second aspect, the present invention relates to a method, wherein instead of an agonist, an antagonist is identified. Thus, such a method of identifying a compound capable of deactivating a G-protein coupled receptor (GPCR) comprises

(a) contacting cell membrane from at least one GPCR expressing cell or cell line with at least one GPCR agonist, at least one kinase and at least one arrestin in a suitable buffer;

(b) contacting at least one test compound with the resulting mixture to allow said test compound to bind to the GPCR and thereby displace any agonist previously bound thereto;

(c) separating the GPCR bound arrestin from the unbound arrestin; and

(d) determining the level of GPCR bound arrestin,

whereby a compound, which is an antagonist of said GPCR, is identified when the level of GPCR bound arrestin is lowered relative to a control.

By “lowered” is meant a level, which is significantly lower than the level that may be determined after step (b), but before the addition of test compound. Thus, the amount of GPCR bound arrestin is reduced after step (c) as compared to the amount bound after step (b). In this context, it is noted that all the methods according to the present invention may include use of a control and/or control measurements, as discussed above.

Agonists may have been obtained by the method described above or are known ligands for the studied receptors, e.g. hormones, amino acids, peptides, proteins or photons.

The details given above regarding a method for identification of agonists according to the present invention are also applicable in this aspect. In particular may the kinase be a G-protein coupled receptor kinase (GRK), such as GRK-2 and/or the arrestin may be β-arrestin.

In one embodiment of the above-described method of identifying an antagonist, this method may also be modified in order to exclude the use of phosphorylating enzyme, which modification is achieved by the use of a mutated arrestin, wherein the mutation renders the arrestin phosphorylation independent as described above. Thus, such a method comprises

(a) contacting cell membrane from at least one GPCR expressing cell or cell line with at least one GPCR agonist and at least one phosphorylation independent arrestin mutant in a suitable buffer;

(b) contacting at least one test compound with the resulting mixture to allow said test compound to bind to the GPCR and thereby displace agonist previously bound thereto;

(c) separating the GPCR bound arrestin mutant from the unbound arrestin mutant; and

(d) determining the level of GPCR bound arrestin,

whereby a compound, which is an antagonist of said GPCR, is identified when the level of GPCR bound arrestin is lowered relative to a control.

The further details regarding this embodiment are as discussed above. In particular, the arrestin mutant may be a β-arrestin mutant, which in turn may be R169E-β-arrestin or 1-382-β-arrestin as described above, and the test compound in step (a) may also furthermore be brought into contact with a kinase for example a G-protein coupled receptor kinase (GRK), such as GRK-2.

In a method according to the present invention for identifying an antagonist, the separation of the GPCR bound arrestin from the unbound arrestin may also be achieved by use of a carrier material capable of binding the cell membranes. Such a method will thus comprise the following steps:

(a) contacting cell membrane from at least one GPCR expressing cell or cell line with at least one GPCR agonist, at least one kinase and at least one arrestin in a suitable buffer;

(b) contacting at least one test compound with the resulting mixture to allow said test compound to bind to the GPCR and thereby displace any agonist previously bound thereto;

(c) contacting the mixture resulting from (c) with carrier material capable of binding said cell membrane(s); and

(d) determining the level of GPCR bound arrestin,

whereby a compound, which is an antagonist of said GPCR, is identified when the level of GPCR bound arrestin is lowered relative to a control.

The further details regarding this embodiment are as discussed above for a method for identifying agonists wherein the separation of the GPCR bound arrestin from the unbound arrestin is achieved by use of a carrier material capable of binding the cell membranes. In particular may the kinase be a G-protein coupled receptor kinase (GRK), such as GRK-2 and/or the arrestin may be β-arrestin.

In one embodiment, the method described above is modified by the use of a mutant arrestin, wherein the mutation renders the arrestin phosphorylation independent as described above. Such a method will thus comprise the following steps:

(a) contacting cell membrane from at least one GPCR expressing cell or cell line with at least one GPCR agonist and at least one phosphorylation independent arrestin mutant in a suitable buffer;

(b) contacting at least one test compound with the resulting mixture to allow said test compound to bind to the GPCR and thereby displace agonist previously bound thereto;

(c) contacting the mixture resulting from (c) with carrier material capable of binding said cell membrane(s); and

(d) determining the level of GPCR bound arrestin,

whereby a compound, which is an antagonist of said GPCR, is identified when the level of GPCR bound arrestin is lowered relative to a control.

The further details regarding this embodiment are as discussed above. In particular, the arrestin mutant may be a β-arrestin mutant, which in turn may be R169E-β-arrestin or 1-382-β-arrestin as described above, and the test compound in step (a) may also furthermore be brought into contact with a kinase for example a G-protein coupled receptor kinase (GRK), such as GRK-2.

Mutant GPCRs, such as constitutively active mutant GPCRs, may also be used in a method according to the present invention. “Constitutively active mutant GPCRs” are GPCRs which has a basal receptor activity even in the absence of ligand binding. Use of such GPCR mutants enables the identification of compounds which down-regulates the activity of the GPCR in a method according to the present invention without the need for a competition assay as described above. Such a method comprises

(a) contacting at least one test compound with a preparation of cell membrane from at least one cell or cell line expressing a constitutively active mutant GPCR, at least one kinase, and at least one arrestin in a suitable buffer;

(b) separating the GPCR bound arrestin from the unbound arrestin; and

(c) determining the level of GPCR bound arrestin,

whereby a compound, which is an antagonist of said GPCR, is identified when the level of GPCR bound arrest in is lowered relative to a control. The further details regarding these embodiment are as discussed above; in particular the kinase may be a G-protein coupled receptor kinase (GRK), such as GRK-2 and/or the arrestin may be β-arrestin. The present invention also encompasses modifications of said methods, wherein the arrestin is a phosphorylation independent arrestin mutant, such as a β-arrestin mutant, for instance R169E-β-arrestin or 1-382-β-arrestin as described above. As described above, a kinase, such as a G-protein coupled receptor kinase (GRK), such as GRK-2, may also be used in methods wherein the arrestin is an phosphorylation independent arrestin mutant as described above. In one embodiment of said methods, the separation of the GPCR bound arrestin from the unbound arrestin may also be achieved by use of a carrier material capable of binding the cell membranes. Such a method comprises

(a) contacting at least one test compound with a preparation of cell membrane from at least one cell or cell line expressing a constitutively active mutant GPCR, at least one kinase, and at least one arrestin in a suitable buffer;

(b) contacting the resulting mixture with carrier material capable of binding said cell membrane(s); and

(c) determining the level of GPCR bound arrest in,

whereby a compound, which is an antagonist of said GPCR, is identified when the level of GPCR bound arrestin is lowered relative to a control. The further details regarding these embodiment are as discussed above. In particular, the carrier material may be provided with wheat germ agglutinate (WGA) to allow binding of cell membrane(s) expressing GPCR(s). In one embodiment of the method according to the present invention, the arrestin used, whether it is arrestin, β-arrestin or a mutant arrestin, is labelled. This will allow for easier detection of the level of GPCR bound arrestin as the level of GPCR bound arrestin will correspond to the level of detected signals emitted from the formed (arrestin-GPCR-carrier) complex. When the arrestin is labelled, the determination of the level of GPCR bound arrestin may thus be performed by detecting signals emitted from the formed (arrestin-GPCR) or (arrestin-GPCR-carrier) complex, whereby a compound which is an agonist of said GPCR is identified when signals are emitted, and whereby an antagonist of said GPCR is identified when a reduction in signal is observed. These complexes are formed when the arrestin are bound to the GPCR and when the GPCR in turn are bound to carrier material, in cases where such carrier material is present. As mentioned previously, the terms “(arrestin-GPCR) complex” and “(arrestin-GPCR-carrier) complex” shall also encompass any complexes comprising β-arrestin or any arrestin mutants, such as R169E-β-arrestin or 1-382-β-arrestin as well as complexes comprising GPCR mutants. By “reduction” is meant a signal, which is significantly lower than the signal that may be detected after step (b), but before the addition of test compound. Thus, the amount of GPCR bound β-arrestin is reduced after step (c) as compared to the amount bound after step (b). In this context, it is noted that all the methods according to the present invention may include use of a control and/or control measurements, as discussed above.

Labelled arrestin may for instance be radioactively labelled and may for instance be made according to the protocol described by Sohlemann et al., Eur. J. Biochem. 232, 464-472 (1995).

In one embodiment of a method according to the present invention, wherein the separation of the GPCR bound arrestin from the unbound arrestin is achieved by use of a carrier material capable of binding the cell membranes, the carrier material also provides the signal which is detected in the last step, which signal is emitted when the label of the arrestin is sufficiently close to the carrier for an interaction between the two. In a further embodiment, the signal emitted by the carrier material, or rather, from the formed (arrestin-GPCR-carrier) complex, is light due to scintillation and the arrestin is radioactively labelled.

Thus, in this embodiment of a method according to the present invention, the detection step is uncomplicated, fast and efficient, since steps for separating GPCR bound arrestin from GPCR unbound arrestin are avoided. The label of the arrestin is advantageously a radioactive label (for a reference to radio labelling and fluorescent labelling, see e.g. Atlas et al., Proc. Natl. Acad. Sci. USA 74, 5490 (1977) and U.S. Pat. No. 5,576,436).

In a still further embodiment of this method, the carrier material comprises scintillation proximity assay (SPA) beads. In such a case, light is emitted when the beads recognize the presence of a radioactively labelled arrestin bound to the receptor, where the receptor in turn has been bound to the beads for instance via a layer of WGA or antibodies as previously described.

The use of the scintillation proximity assay (SPA) to study enzyme reactions and receptor-ligand-interactions is a relatively new type of assay which however is well known in the art by now (see for example Cook, N. D., Drug Discovery Today, 1(7), 287-294 (1996): Scintillation proximity assay—a versatile high throughput screening technology).

In the present context, the separation of a desired signal from disturbing background noise may e.g. be performed by first measuring the signal emitted of the components without the test compound in order to establish a value of the background. Signals are then detected for determining the level of GPCR bound arrestin, and from that detection the background value is subtracted. The method used for the detection may e.g. depend on the carrier material-label used.

As mentioned above, the skilled person will be able to select a suitable methodology to separate the signal emitted from formed (arrestin-GPCR) or (arrestin-GPCR-carrier) complex as an indication of bound β-arrestin from any disturbing background noise.

The methods according to the present invention are useful for screening large numbers of test compounds in order to identify agonists or antagonists for a G-protein coupled receptor, which agonists or antagonists are possible drugs. Such screening may e.g. be performed in microplates using techniques and equipment well known to those of skill in screening technology. Buffers for this purpose are also well known in the field, as illustrated below in the section “Experimental”. Drugs may be identified which are useful in a vast number of clinical conditions, including e.g. diabetes, metabolic diseases, and problems associated with vasodilation, cardiac disorders, bronchodilation, cancer and endocrine secretion (see e.g. Lefkowitz et al., Ann. Rev. Biochem. 52,159 (1983)).

In a most advantageous embodiment of a method according to the present invention, the method according to the present invention is a high throughput screening (HTS) assay, enabling the testing of a large number of samples in a short time. For a reference to such methods, see e.g. Picardo, M., Hughes, K. T., High Throughput Screening. The discovery of Bioactive Substances, pp. 307-316, Ed Devlin, J. P., 1997.

In a third aspect, the present invention relates to the use of a compound identified according to a method according to the present invention as a therapeutically effective substance In a fourth aspect, the present invention relates to the use of a compound identified according to a method according to the invention as a lead compound in drug design, wherein the structure and/or biological properties of said compound are modified in order to provide a therapeutically effective substance. Such modification may e.g. be performed in order to eliminate or reduce undesired side effects in a human, enhance the biological properties thereof, facilitate the preparation thereof, enhance the specificity of the action etc. Thus, in a fifth aspect, the present invention relates to a method for producing a pharmaceutical preparation comprising a method according to the present invention, wherein the identified compound is mixed with a pharmaceutically acceptable carrier or transporter. In a sixth aspect, the present invention relates to a method for producing a pharmaceutical preparation comprising a method according to the present invention wherein the structure and/or biological properties of the identified compound is modified in order to provide a therapeutically effective substance. The further preparation of pharmaceutical preparations comprising a compound identified by use of a method according to the present invention may take place according to conventional techniques, e.g. as described in Remington: The Science and Practise of Pharmacy, 19^{th} Ed. 1995. The pharmaceutical preparations may appear in conventional forms, for example capsules, tablets, aerosols, solutions, suspensions or topical applications. Typical pharmaceutical preparations comprise a compound identified by use of a method according to the present invention or a pharmaceutically acceptable basic addition salt or prodrug or hydrate thereof, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical preparations may be used. For example, the compound identified by use of a method according to the present invention will usually be mixed with a carrier, or diluted by a carrier, or enclosed within a carrier, which may be in the form of a ampoule, capsule, sachet, paper, or other container. When the carrier serves as a diluent, it may be solid, semi-solid, or liquid material, which acts as a vehicle, excipient, or medium for the active compound. The compound identified by use of a method according to the present invention can be adsorbed on a granular solid container for example in a sachet. Some examples of suitable carriers are water, salt solutions, alcohols, polyethylene glycols, polyhydroxyethoxylated castor oil, peanut oil, olive oil, gelatine, lactose, terra alba, sucrose, dextrin, magnesium carbonate, sugar, cyclodextrin, amylose, magnesium stearate, talc, gelatine, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the present invention may be formulated so as to provide quick, sustained, or delayed release of the active ingredient after administration to the patient by employing procedures well known in the art.

The pharmaceutical preparations can be sterilized and mixed, if desired, with auxiliary agents, emulsifiers, salt for influencing osmotic pressure, buffers and/or colouring substances and the like, which do not deleteriously react with the active compounds.

The route of administration may be any route, which effectively transports the compound identified by use of a method according to the present invention to the appropriate or desired site of action, such as oral, nasal, pulmonary, buccal, subdermal, intradermal, transdermal or parenteral e.g. rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound identified by use of a method according to the present invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the compound identified by use of a method according to the present invention dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet which may be prepared by conventional tabletting techniques may contain:

| Core: | | |
|---|---|---|
| Active compound (as free compound or salt thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

The dosage and administration form of the final product will depend on the condition to be treated as well as on the age, sex, weight etc of the patient to be treated.

### EXAMPLES

The present examples are provided for illustrative purposes only and shall not be construed as limiting the scope of the present invention as defined by the appended claims. All references given below and elsewhere in the present specification are hereby included herein by reference.

The assays described in the examples below are performed in 96 or 384 well microplates. Alternatively, other platforms such as 1536 well micro plates can be used, provided the added volumes and concentrations are corrected accordingly. Other platforms can also be used, such as for instance a microfuge tube or a test tube. All components are dissolved or suspended in assay buffer as follows: 50 mM Tris-HCl, pH 7.5, 50 mM potassium acetate, 0.5 mM MgCl₂, 1 mM DTT, and 0.5 mM ATP (ATP is not necessary when phosphorylation independent β-arrestin is used). The sample comprising the test compound may comprise several test compounds to be added to the same well, for instance when combinatorial chemical libraries are being examined. GRK used in these experiments is expressed in Sf9 cells and purified to apparent homogeneity as described in Söhlemann et al., FEBS Letters 324, 59-62 (1993). Labelled β-arrestin is expressed in Sf9 cells according to the protocol described by Sohlemann et al., Eur. J. Biochem. 232, 464-472 (1995).

### Example 1

### Identification of Agonists Using Cell Membrane from BHK Cells

To each well of a 96 well plate is added:

5 μl sample which comprises a test compound is added to each well at 0.6 mg/ml. The test compound has been dissolved in DMSO and originates from a compound library.

50 μl Dopamin D1a receptor containing cell membranes at 0.1 mg membrane/ml. The cell membranes are prepared from BHK cells expressing the receptor. The membrane preparation and the two-part reconstitution (membrane fusion) of plasma membrane were made according to the protocol described by Cerione et al. 1983 (Nature 306, 562-566).

50 μl bovine GRK-2 at 10 μM.

50 μl radio labelled, bovine β-arrestin at 100000 cmp/10 μl, ¹²⁵I labelled β-arrestin is used.

50 μl wheat germ agglutinate (WGA) coated scintillation proximity assay (SPA) (Amersham Pharmacia Biotech, Cardiff, Wales) beads at 15 mg/ml.

The reconstituted receptors (50 nM) is phosphorylated for 30 min at 30° C. by purified GRK (4 nM) in 20 mM Tris/HCl, pH 7.6, 2 mM EDTA and 8 mM MgCl₂ in the presence of 200 μM ATP, 10 μM agonist and 500 nM G protein βγ subunits.

The resulting mixture is then incubated for one hour at room temperature with shaking, during which time complexes are allowed to form between activated and phosphorylated receptors and β-arrestin.

In order to detect agonists of the Dopamin D1a receptor, the plates are read/counted in a TopCount (from Packard Instrument Company). Light is detected as an indication of the presence of agonist, since labelled β-arrestin binds to activated and phosphorylated receptor, i.e., to receptor activated by a test compound capable of acting as an agonist. The receptor is in turn bound to SPA beads, thus enabling light emission as a result of the interaction bead-radioactive label.

Accordingly, test compounds present in the samples added to the wells from which light is detected are selected for further investigation as possible drugs.

### Example 2

### Identification of Agonists Using a β-arrestin Mutant and Cell Membrane from BHK Cells

To each well of a 384 well plate is added:

1 μl sample which comprises a test compound is added at 0.6 mg/ml. The test compound is from a compound library and has been dissolved in water.

10 μl GPCR (G-protein coupled receptor) containing cell membranes at 0.1 mg membrane/ml. Cell membranes are prepared from cells expressing the GPCR/BHK cells?/ Cells are acquired from Euroscreen (www.euroscreen.be/UGARF.htm). The membrane preparation and the two-part reconstitution (membrane fusion) of plasma membrane were made according to the protocol described by Cerione et al. 1983 (Nature 306, 562-566).

10 μl radio labelled β-arrestin R169E (wherein amino acid no. 169, arginine, has been changed to a glutamic acid), 100000 cmp/10 μl, ¹²⁵I labelled β-arrestin is used.

10 μl wheat germ agglutinate (WGA) coated Leadseeker beads (from Amersham Pharmacia Biotech, APB) at 15 mg/ml.

The resulting mixture is then incubated for one hour at room temperature, during which time complexes are allowed to form between activated receptors and phosphorylation independent β-arrestin.

The plate is counted/read using a Leadseeker CCD camera (from Amersham Pharmacia Biotech, APB). As in Ex 1 above, light emission is an indication of an agonist. Accordingly, test compounds added to the wells from which light is emitted are selected for further investigation as possible drugs.

### Example 3

### Identification of Agonists Using Cell Membrane from Yeast Cells

To each well of a 96 well plate is added:

5 μl sample which comprises a test compound is added to each well at 0.6 mg/ml. The test compound has been dissolved in DMSO and originates from a compound library.

50 μl Dopamin D1a receptor containing cell membranes at 0.1 mg membrane/ml. The cell membranes are prepared from Saccharomyces cerevisiae cells genetically modified to express the receptor. Cell membranes are prepared according to the protocols described by Menendez et al., Anal. Biochem. 230, 308-314 (1995). Electron microscopi is used for confirming that plasma membrane sheets and unsealed vesicles are obtained with this method.

50 μl bovine GRK-2 at 10 μM.

50 μl radio labelled, bovine β-arrestin at 100000 cmp/10 μl, ¹²⁵I labelled β-arrestin is used.

50 μl wheat germ agglutinate (WGA) coated scintillation proximity assay (SPA) (Amersham Pharmacia Biotech, Cardiff, Wales) beads at 15 mg/ml.

The resulting mixture is then incubated for one hour at room temperature with shaking, during which time complexes are allowed to form between activated and phosphorylated receptors and β-arrestin.

The plate is counted/read using a Leadseeker CCD camera (from Amersham Pharmacia Biotech, APB). As in Ex 1 above, light emission is an indication of an agonist. Accordingly, test compounds added to the wells from which light is emitted are selected for further investigation as possible drugs

### Example 4

### Identification of Agonists Using a β-arrestin Mutant and Cell Membrane from Yeast Cells

To each well of a 384 well plate is added:

1 μl sample which comprises a test compound is added at 0.6 mg/ml. The test compound is from a compound library and has been dissolved in water.

10 μl GPCR (G-protein coupled receptor) containing cell membranes at 0.1 mg membrane/ml. Cell membranes are prepared from Saccharomyces cerevisiae cells genetically modified to express the receptor. Cell membranes are prepared according to the protocols described by Menendez et al., Anal. Biochem. 230, 308-314 (1995). Electron microscopi is used for confirming that plasma membrane sheets and unsealed vesicles are obtained with this method.

10 μl radio labelled β-arrestin R169E (wherein amino acid no. 169, arginine, has been changed to a glutamic acid), 100000 cmp/10 μl, ¹²⁵I labelled β-arrestin is used.

10 μl wheat germ agglutinate (WGA) coated Leadseeker beads (from Amersham Pharmacia Biotech, APB) at 15 mg/ml.

The resulting mixture is then incubated for one hour at room temperature, during which time complexes are allowed to form between activated receptors and phosphorylation independent β-arrestin.

The plate is counted/read using a Leadseeker CCD camera (from Amersham Pharmacia Biotech, APB). As in Ex 1 above, light emission is an indication of an agonist. Accordingly, test compounds added to the wells from which light is emitted are selected for further investigation as possible drugs.

### Example 5

### Identification of an Antagonist Using Cell Membrane from BHK Cells

To each well of a 384 well plate is added:

10 μl agonist in a concentration sufficient to provide activation of essentially all of the receptors present in each well.

1 μl sample which comprises a test compound is added at 0.6 mg/ml. The test compound is from a compound library and has been dissolved in water.

10 μl GPCR (G-protein coupled receptor) containing cell membranes at 0.1 mg membrane/mi. Cell membranes are prepared from cells expressing the GPCR under investigation. Cells are acquired from Euroscreen (www.euroscreen. be/UGARF.htm). The membrane preparation and the two-part reconstitution (membrane fusion) of plasma membrane were made according to the protocol described by Cerione et al. 1983 (Nature 306, 562-566).

10 μl bovine GRK-2 at 10 μM.

10 μl radio labelled β-arrestin, 100000 cmp/1 82 l, ¹²⁵I labelled β-arrestin is used.

10 μl wheat germ agglutinate (WGA) coated Leadseeker beads (from Amersham Pharmacia Biotech, APB) at 15 mg/ml.

The reconstituted receptors (50 nM) is phosphorylated for 30 min at 30° C. by purified GRK (4 nM) in 20 mM Tris/HCl, pH 7.6, 2 mM EDTA and 8 mM MgCl₂ in the presence of 200 μM ATP, 10 μM agonist and 500 nM G protein βγ subunits.

The resulting mixture is then incubated for one hour at room temperature, during which time complexes are allowed to form between activated and phosphorylated receptors and β-arrestin.

The plate is counted/read in a Leadseeker as described above. A reduction of light emission is here an indication of a test compound capable of acting as an antagonist. Thus, test compounds from which such a reduction is detected are selected for further investigation as drugs.

### Example 6

### Identification of an Antagonist Using a β-arrestin Mutant and Cell Membrane from BHK Cells

To each well of a 384 well plate is added:

10 μl agonist in a concentration sufficient to activate a detectable amount of the receptors present in each well.

1 μl sample which comprises a test compound is added at 0.6 mg/ml. The test compound originates from a compound library and has been dissolved in DMSO.

10 μl GPCR (G-protein coupled receptor) containing cell membranes at 0.1 mg membrane/ml. Cell membranes are prepared from cells expressing the GPCR under investigation. Cells membranes are acquired from Euroscreen (www.eurbscreen.be/UGARF.htm). The cell membranes are prepared using standard methods. The membrane preparation and the two-part reconstitution (membrane fusion) of plasma membrane were made according to the protocol described by Cerione et al. 1983 (Nature 306, 562-566).

10 μl radio labelled β-arrestin R169E, 100000 cmp/10 μl, ¹²⁵I labelled β-arrestin is used.

10 μl wheat germ agglutinate (WGA) coated Leadseeker beads at 15 mg/ml.

The resulting mixture is then incubated for one hour at room temperature, during which time complexes are allowed to form between activated receptors and phosphorylation independent β-arrestin.

The plate is counted/read in a Leadseeker as described above. As in Ex 5, a reduction in light emission is an indication of a test compound capable of acting as an antagonist. Thus, test compounds from which such a reduction is observed are selected for use as lead compounds in the development of drugs.

### Example 7

### Identification of an Antagonist Using Cell Membrane from Yeast Cells

To each well of a 384 well plate is added:

10 μl agonist in a concentration sufficient to provide activation of essentially all of the receptors present in each well.

1 μl sample which comprises a test compound is added at 0.6 mg/ml. The test compound is from a compound library and has been dissolved in water.

10 μl GPCR (G-protein coupled receptor) containing cell membranes at 0.1 mg membrane/ml. The cell membranes are prepared from Saccharomyces cerevisiae cells genetically modified to express the receptor under investigation. Cell membranes are prepared according to the protocols described by Menendez et al., Anal. Biochem. 230, 308-314 (1995). Electron microscopi is used for confirming that plasma membrane sheets and unsealed vesicles are obtained with this method.

10 μl bovine GRK-2 at 10 μM.

10 μl radio labelled β-arrestin, 100000 cmp/10 μl, ¹²⁵I labelled -arrestin is used.

10 μl wheat germ agglutinate (WGA) coated Leadseeker beads (from Amersham Pharmacia Biotech, APB) at 15 mg/ml.

The reconstituted receptors (50 nM) is phosphorylated for 30 min at 30° C. by purified GRK (4 nM) in 20 mM Tris/HCl, pH 7.6, 2 mM EDTA and 8 mM MgCl₂ in the presence of 200 μM ATP, 10 μM agonist and 500 nM G protein βγ subunits.

The resulting mixture is then incubated for one hour at room temperature, during which time complexes are allowed to form between activated and phosphorylated receptors and β-arrestin.

The plate is counted/read in a Leadseeker as described above. As in Ex 5, a reduction in light emission is an indication of a test compound capable of acting as an antagonist. Thus, test compounds from which such a reduction is observed are selected for use as lead compounds in the development of drugs.

### Example 8

### Identification of an Antagonist Using a β-arrestin Mutant and Cell Membrane from Yeast Cells

To each well of a 384 well plate is added:

10 μl agonist in a concentration sufficient to activate a detectable amount of the receptors present in each well.

1 μl sample which comprises a test compound is added at 0.6 mg/ml. The test compound originates from a compound library and has been dissolved in DMSO.

10 μl GPCR (G-protein coupled receptor) containing cell membranes at 0.1 mg membrane/ml. The cell membranes are prepared from Saccharomyces cerevisiae cells genetically modified to express the receptor under investigation. Cell membranes are prepared according to the protocols described by Menendez et al., Anal. Biochem. 230, 308-314 (1995). Electron microscopi is used for confirming that plasma membrane sheets and unsealed vesicles are obtained with this method.

10 μl radio labelled β-arrestin R169E, 100000 cmp/10 μl, ¹²⁵I labelled β-arrestin is used.

10 μl wheat germ agglutinate (WGA) coated Leadseeker beads at 15 mg/ml.

The resulting mixture is then incubated for one hour at room temperature, during which time complexes are allowed to form between activated receptors and phosphorylation independent β-arrestin.

The plate is counted/read in a Leadseeker as described above. As in Ex 5, a reduction in light emission is an indication of a test compound capable of acting as an antagonist. Thus, test compounds from which such a reduction is observed are selected for use as lead compounds in the development of drugs.

What is claimed is:

1. A method of identifying a compound capable of initiating the signalling of a G-protein coupled receptor (GPCR), which method comprises
(a) contacting at least one test compound with cell membrane from at least one GPCR expressing cell or cell line, at least one kinase, and at least one arrestin in a suitable buffer;
(b) separating the GPCR bound arrestin from the unbound arrestin; and
(c) determining the level of GPCR bound arrestin,
whereby a compound which is an agonist of said GPCR is identified when the level of GPCR bound arrestin is raised relative to a control.

2. A method according to claim 1, wherein said kinase is a G-protein coupled receptor kinase (GRK).

3. A method according to claim 1, wherein said arrestin is β-arrestin.

4. A method of identifying a compound capable of initiating the signalling of a G-protein coupled receptor (GPCR), which method comprises
(a) contacting at least one test compound with cell membrane from at least one GPCR expressing cell or cell line and at least one phosphorylation-independent arrestin mutant;
(b) separating the GPCR bound arrestin mutant from the unbound arrestin mutant; and
(c) determining the level of GPCR bound arrestin mutant,
whereby a compound which is an agonist of said GPCR is identified when the level of GPCR bound arrestin mutant is raised relative to a control.

5. A method according to claim 4, wherein said arrestin mutant is a β-arrestin mutant.

6. A method according to claim 5, wherein said β-arrestin mutant is R169E-β-arrestin or 1-382-β-arrestin.

7. A method according to claim 4, wherein the test compound in step (a) is furthermore brought into contact with a kinase.

8. A method according to claim 7, wherein said kinase is a G-protein coupled receptor kinase (GRK).

9. A method according to claim 1, wherein the arrestin is a labelled arrestin and the determination of the level of GPCR bound arrestin is performed by detecting signals emitted from the formed (arrestin-GPCR) complex; whereby a compound which is an agonist of said GPCR is identified when signals are emitted.

10. A method of identifying a compound capable of initiating the signalling of a G-protein coupled receptor (GPCR), which method comprises
(a) contacting at least one test compound with cell membrane from at least one GPCR expressing cell or cell line; at least one kinase; and at least one arrestin in a suitable buffer;
(b) contacting the resulting mixture with carrier material capable of binding said cell membrane(s); and
(c) determining the level of GPCR bound arrestin,
whereby a compound which is an agonist of said GPCR is identified when the level of GPCR bound arrestin is raised relative to a control.

11. A method according to claim 10, wherein said kinase is a G-protein coupled receptor kinase (GRK).

12. A method according to claim 10, wherein said arrestin is β-arrestin.

13. A method of identifying a compound capable of initiating the signalling of a G-protein coupled receptor (GPCR), which method comprises
(a) contacting at least one test compound with cell membrane from at least one GPCR expressing cell or cell line and at least one phosphorylation independent arrestin mutant in a suitable buffer;
(b) contacting the resulting mixture with carrier material capable of binding said cell membrane(s); and
(c) determining the level of GPCR bound arrestin,
whereby a compound which is an agonist of said GPCR is identified when the level of GPCR bound arrestin mutant is raised relative to a control.

14. A method according to 13, wherein said arrestin mutant is a β-arrestin mutant.

15. A method according to claim 14, wherein said β-arrestin mutant is R169E-β-arrestin or 1-382-β-arrestin.

16. A method according to claim 13 wherein the test compound in step (a) is furthermore brought into contact with a kinase.

17. A method according to claim 16, wherein said kinase is a G-protein coupled receptor kinase (GRK), such as GRK-2.

18. A method according to claim 10, wherein the arrestin is a labelled arrestin and the determination of the level of GPCR bound arrestin is performed by detecting signals emitted from the formed (arrestin-GPCR-carrier) complex; whereby a compound which is an agonist of said GPCR is identified when signals are emitted.

19. A method according to claim 18, wherein the signal emitted by the formed (arrestin-GPCR-carrier) complex is light due to scintillation and the arrestin is radioactively labelled.

20. A method according to claim 19, wherein the carrier material comprises scintillation proximity assay (SPA) beads.

21. A method according to claim 10, wherein the carrier material is provided with wheat germ agglutinate (WGA) to allow binding of cell membrane(s) expressing GPCR(s).

22. A method of identifying a compound capable of deactivating a G-protein coupled receptor (GPCR), which method comprises
(a) contacting cell membrane from at least one GPCR expressing cell or cell line with at least one GPCR agonist, at least one kinase and at least one arrestin in a suitable buffer;
(b) contacting at least one test compound with the resulting mixture to allow said test compound to bind to the GPCR and thereby displace any agonist previously bound thereto;
(c) separating the GPCR bound arrestin from the unbound arrestin; and
(d) determining the level of GPCR bound arrestin,
whereby a compound which is an antagonist of said GPCR is identified when the level of GPCR bound arrestin is lowered relative to a control.

23. A method according to claim 22, wherein said kinase is a G-protein coupled receptor kinase (GRK), such as GRK-2.

24. A method according to claim 22, wherein said arrestin is β-arrestin.

25. A method of identifying a compound capable of deactivating a G-protein coupled receptor (GPCR), which method comprises
(a) contacting cell membrane from at least one GPCR expressing cell or cell line with at least one GPCR agonist and at least one phosphorylation independent arrestin mutant in a suitable buffer;
(b) contacting at least one test compound with the resulting mixture to allow said test compound to bind to the GPCR and thereby displace agonist previously bound thereto;
(c) separating the GPCR bound arrestin mutant from the unbound arrestin mutant; and
(d) determining the level of GPCR bound arrestin,
whereby a compound which is an antagonist of said GPCR is identified when the level of GPCR bound arrestin is lowered relative to a control.

26. A method according to claim 25, wherein said arrestin mutant is a β-arrestin mutant.

27. A method according to claim 26, wherein said β-arrestin mutant is R169E-β-arrestin or 1-382-β-arrestin.

28. A method according to claim 25 wherein the test compound in step (a) is furthermore brought into contact with a kinase.

29. A method according to claim 28, wherein said kinase is a G-protein coupled receptor kinase (GRK), such as GRK-2.

30. A method according to claim 22, wherein the arrestin is a labelled arrestin and the determination of the level of GPCR bound arrestin is performed by detecting signals emitted from the formed (arrestin-GPCR) complex; whereby a compound which is an antagonist of the GPCR used is identified when a reduction in signal is detected.

31. A method of identifying a compound capable of deactivating a G-protein coupled receptor (GPCR), which method comprises
(a) contacting cell membrane from at least one GPCR expressing cell or cell line with at least one GPCR agonist, at least one kinase and at least one arrestin in a suitable buffer;
(b) contacting at least one test compound with the resulting mixture to allow said test compound to bind to the GPCR and thereby displace any agonist previously bound thereto;
(c) contacting the mixture resulting from (c) with carrier material capable of binding said cell membrane(s); and
(d) determining the level of GPCR bound arrestin,
whereby a compound which is an antagonist of said GPCR is identified when the level of GPCR bound arrest in is lowered relative to a control.

32. A method according to claim 31, wherein the kinase is a G-protein coupled receptor kinase (GRK), such as GRK-2.

33. A method according to claim 31, wherein the arrestin is β-arrestin.

34. A method of identifying a compound capable of deactivating a G-protein coupled receptor (GPCR), which method comprises
(a) contacting cell membrane from at least one GPCR expressing cell or cell line with at least one GPCR agonist and at least one phosphorylation independent arrestin mutant in a suitable buffer;
(b) contacting at least one test compound with the resulting mixture to allow said test compound to bind to the GPCR and thereby displace agonist previously bound thereto;
(c) contacting the mixture resulting from (c) with carrier material capable of binding said cell membrane(s); and
(d) determining the level of GPCR bound arrestin,
whereby a compound which is an antagonist of said GPCR is identified when the level of GPCR bound arrestin is lowered relative to a control.

35. A method according to claim 34, wherein said arrestin mutant is a β-arrestin mutant.

36. A method according to claim 35, wherein said β-arrestin mutant is R169E-β-arrestin or 1-382-β-arrestin.

37. A method according to claim 34 wherein the test compound in step (a) is furthermore brought into contact with a kinase.

38. A method according to claim 37, wherein said kinase is a G-protein coupled receptor kinase (GRK), such as GRK-2.

39. A method according to claim 31, wherein the arrestin is a labelled arrestin and the determination of the level of GPCR bound arrestin is performed by detecting signals emitted from the formed (arrestin-GPCR-carrier) complex; whereby a compound which is an antagonist of the GPCR used is identified when a reduction in signal is detected.

40. A method according to claim 39, wherein the signal emitted by the formed (arrestin-GPCR-carrier) complex is light due to scintillation and the arrestin is radioactively labelled.

41. A method according to claim 40, wherein the carrier material comprises scintillation proximity assay (SPA) beads.

42. A method according to claim 31, wherein the carrier material is provided with wheat germ agglutinate (WGA) to allow binding of cell membrane(s) expressing GPCR(s).

43. A method according to claim 1, which is a high throughput screening method.

44. Use of a compound identified according to a method as defined in any of claims 1 to 43 as a therapeutically effective substance.

45. Use of a compound identified according to a method as defined in any of claims 1 to 43 as a lead compound in drug design, wherein structure and/or biological properties of said compound are modified in order to provide a therapeutically effective substance.

46. A method for producing a pharmaceutical preparation comprising (i) identifying a compound using a method according to claim 1, and (ii) mixing the identified compound with a pharmaceutically acceptable carrier.

47. A method for producing a pharmaceutical preparation comprising (i) modifying the structure and/or biological properties of a compound identified using a method according to claim 1, (ii) mixing the modified comopound with a pharmaceutically acceptable carrier.
